(19) United States
(12) Patent Application Publication
(10) Pub. No.: US 2018/0235704 A1
(43) Pub. Date: Aug. 23, 2018

(54) CONFIGURING A STEREO-ELECTROENCEPHALOGRAPHY PROCEDURE

(71) Applicant: THE CLEVELAND CLINIC FOUNDATION, Cleveland, OH (US)

(72) Inventors: Jorge A. Gonzalez-Martinez, Cleveland, OH (US); Imad Najm, Cleveland, OH (US); Zhong Wang, Cleveland, OH (US)

(21) Appl. No.: 15/901,310

(22) Filed: Feb. 21, 2018

Related U.S. Application Data
(60) Provisional application No. 62/461,396, filed on Feb. 21, 2017.

(51) Int. Cl. A61B 34/10; A61B 5/0478; A61B 5/00; A61B 90/00; A61B 5/16

(52) U.S. Cl. CPC A61B 34/10; A61B 5/0478; A61B 5/6868; A61B 90/37; A61B 2562/0209; A61B 5/4094; A61B 2034/107; A61B 2560/063; A61B 5/16

(57) ABSTRACT

Systems and methods for configuring a stereo-electroencephalography to target specific brain regions that are part of large scale neural networks responsible for neurological disorders are described. A hypothesis related to a large scale neural network responsible for a neurological disorder within patient's brain can be received. Coordinates for implantation of at least two depth electrodes in the patient's brain can be determined based on the hypothesis. Electrodes to be implanted can be selected for each of the coordinates. The electrodes can be designed and used to target these specific brain regions, including an insula electrode, a parietal-frontal electrode, a hippocampo electrode, and a perisylvian electrode. A visualization that includes the coordinates and the selected electrodes can be displayed, so that a surgeon can implant the selected electrodes into the patient's brain according to the coordinates to test the hypothesis.

### CROSS-REFERENCE TO RELATED APPLICATIONS

This application claims the benefit of U.S. Provisional Application No. 62/461,396, filed Feb. 21, 2017, entitled STEREOTACTIC COORDINATES AND ELECTRODE DESIGN FOR TARGETING LARGE-SCALE NEURONAL NETWORKS, which is incorporated herein by reference in its entirety.

### TECHNICAL FIELD

The present disclosure relates generally to stereo-electroencephalography (SEEG) and, more specifically, to systems and methods for configuring a SEEG procedure.

### BACKGROUND

Epilepsy refers to a group of neurological disorders characterized by recurring seizures resulting from excessive and abnormal neuronal activity from neuronal networks within the brain. Clinical experience indicates that the earliest sign or symptom is the best marker of seizure onset. However, sensory symptoms can be hidden or not communicated by the patient during the seizure (loss of contact, brisk occurrence of motor signs); this is why electrical patterns that indicate the ictal structure are at least as important the seizure onset. In fact, the ictal structure gives clues about pathways of propagation, which can be used for epileptic zone (EZ) localization. A seizure structure may be compared to a string of characters used to write a language. A sequence of characters that defines the string supports a “meaning” in human language or a “code” in computer programming. Similarly, the intricate string of symptoms and signs that characterizes a seizure is the behavioral expression of an ordered temporal-spatial dynamic of the ictal discharge in the brain. Understanding the syntax of a semiological sequence is the way to access its neural meaning. Deciphering seizure signification leads to representing the seizure in the neural network anatomy where this functional abnormal organization is implemented. This is the basis of stereo-electroencephalography (SEEG).

SEEG is a minimally invasive technique that allows for three-dimensional (3D) sampling of cortical and subcortical deep structures. However, the location of the SEEG electrodes is of utmost importance, such that an efficient strategy needs to be implemented to map the neuronal networks involved in the seizures with the minimal number of intracranial electrodes. Accordingly, before conducting SEEG, an intracranial electrode implantation plan must be developed, which requires a clear formulation of a specific anatomical and electrophysiological hypothesis for the patient. However, currently, there is no standard by which to define such a hypothesis and no specialized intracranial electrodes that exist to facilitate investigation of the hypothesis.

### SUMMARY

The present disclosure relates generally to stereo-electroencephalography (SEEG) and, more specifically, to systems and methods for configuring a SEEG procedure.

In one aspect, the present disclosure includes a method for configuring a stereo-electroencephalography procedure to target specific brain regions that are part of large scale neural networks responsible for neurological disorders. The method can include steps that are executed by a system comprising a processor. The steps can include receiving a hypothesis related to a large scale neural network responsible for a neurological disorder within patient's brain; determining coordinates for implantation of at least two depth electrodes in the patient's brain based on the hypothesis; selecting electrodes to be implanted for each of the coordinates, wherein the electrodes comprise at least one of an insula electrode, a parietal-frontal electrode, a hippocampo electrode, or a perisylvian electrode, and displaying a visualization comprising the coordinates and the selected electrodes. A surgeon can implant the selected electrodes into the patient's brain according to the coordinates to test the hypothesis.

In another aspect, the present disclosure includes a system that configures a stereo-electroencephalography procedure to target specific brain regions that are part of large-scale neural networks responsible for neurological disorders. The system can include a memory storing instructions; and a processor to execute the instructions to perform operations. The operations can include receive a hypothesis related to a large scale neural network responsible for a neurological disorder within patient's brain; determine coordinates for implantation of at least two depth electrodes in the patient's brain based on the hypothesis; select electrodes to be implanted for each of the coordinates, wherein the electrodes comprise at least one of an insula electrode, a parietal-frontal electrode, a hippocampo electrode, or a perisylvian electrode, and display a visualization comprising the coordinates and the selected electrodes. A surgeon implants the selected electrodes into the patient's brain according to the coordinates to test the hypothesis.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram of a system that configures a stereo-electroencephalography procedure to target specific brain regions that are part of large-scale neural networks responsible for neurological disorders in accordance with an aspect of the present invention;

FIG. 2 is diagram of an example of a computing device, which can be used in the system of FIG. 1;

FIG. 3 is an illustration of different depth electrodes that can be chosen by the system of FIG. 1;

FIG. 4 is a process flow diagram illustrating a method for configuring a stereo-electroencephalography procedure to target specific brain regions that are part of large scale neural networks responsible for neurological disorders according to another aspect of the present disclosure;

FIG. 5 is an illustration of an example implantation plan for a temporal lobe-limbic network exploration;

FIG. 6 is an illustration of an example implantation plan for a frontal-parietal network exploration;

FIG. 7 is an illustration of an example implantation plan for a peri-rolandic exploration; and

FIG. 8 is an illustration of an example implantation plan for an occipital-parietal-temporal network exploration.

### DETAILED DESCRIPTION

### I. Definitions

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “hypothesis” can refer to a clear formulation of a theoretical neuronal network to be investigated using stereo-electroencephalography (SEEG). The hypothesis can be used to determine sites for implantation of medical devices. For example, the hypothesis can be defined based on electro-clinical data from a subject, imaging data of the subject, and/or neurophysiological data from the subject.

As used herein, the term “neuronal network” can refer to a group of cells in the brain that are related to a medical condition. For example, a neuronal network related to epilepsy can include cells responsible for the start and propagation of a seizure. The terms “neural network” and “neuronal network” can be used interchangeably herein.

As used herein, the term “SEEG” can refer to a minimally-invasive procedure where a number of medical devices are implanted through small openings in the skull in a stereotaxic manner to predetermined locations to study properties of the locations.

As used herein, the terms “stereotactic” and “stereotaxic” can refer to a technique for locating one or more points inside a patient's body (e.g., the brain) using an external, three-dimensional frame of reference based on a three-dimensional coordinate system.

As used herein, the term “three dimensional coordinate system” can refer to a three-dimensional frame of reference that guides a stereotactic procedure. For example, the patient's brain can be depicted in one or more cross sections, each in reference to a two coordinate frame, so that each cross section can be assigned a range of three coordinate numbers that can be used for positioning a medical device within the patient's brain. One example of a three dimensional coordinate system is a Talairach grid.

As used herein, the term “Talairach grid” can refer to a three-dimensional coordinate system used to map the location of brain structures independent from individual differences in the size and overall shape of the brain. The grid system is defined by making two anchors, the anterior commissure and the posterior commissure, lie on a horizontal line. Distances are measured from the anterior commissure as the origin. The y-axis points posterior and anterior to the commissures, the left and right is the x-axis, and the z-axis is the ventral-dorsal (down and up) directions.

As used herein, the term “coordinate numbers” or “coordinates” can refer to three dimensions in the three dimensional coordinate system that can be used to identify a target area. For example, in a Cartesian coordinate system, the coordinates can include an x-value that corresponds to a latero-lateral dimension, a y-value that corresponds to a dorso-ventral dimension, and a z-value that corresponds to a rostero-caudal dimension. As another example, in a polar coordinate system, the coordinates can include an angle coordinate, a depth coordinate, and an anterio-posterior location coordinate.

As used herein, the term “target area” can refer to a section of a patient's brain that includes the neuronal network and can be identified and/or localized with coordinates within the three-dimensional coordinate system. For example, a medical instrument can be implanted to the target area in a stereotactic procedure.

As used herein, the term “stereotactic device” can refer to a mechanical apparatus that can fix or hold a patient's head in a position in reference to the coordinate system to enable implantation of the medical device into the target area. The stereotactic device can attach to a frame that fixes the patient's head in the position in reference to the coordinate system.

As used herein, the term “neuronal network” can relate to a group of cells within the brain that are responsible for modulating and organizing specific medical conditions.

As used herein, the term “medical condition” can refer to the occurrence of one or more pathological manifestations. For example, the medical condition can be a neurological disorder related to epileptic activity, neuropsychological and/or psychiatric behavior (like depression, anxiety, obsessive compulsiveness, addiction, and the like), abnormalities related to motor function, sensory function, language function, behavior function, memory, vision, and high level cognitive function. The terms “neurological disorder” and “medical condition” can be used interchangeably herein.

As used herein, the term “intracranial implantation” can refer to an act of placing a medical device at a predetermined location within a target area along a trajectory. As an example, the intracranial implantation can be achieved using the guidance of a stereotactic device.

As used herein, the term “trajectory” generally relates to a predefined path followed for intracranial implantation.

As used herein, the term “oblique” can pertain to or involve axes that are not perpendicular to each other.

As used herein, the term “orthogonal” can pertain to or involve axes that are perpendicular to each other.

As used herein, the term “medical device” can refer to something that is placed or implanted within the target area during an intracranial implantation. The medical device can be used to modulate, study, and/or map one or more neuronal networks. Examples of the medical device can include one or more of: an electrode (e.g., recording and/or stimulating), an injection probe (e.g., virus injection, DNA/RNA injection, dye, tracer, etc.), a biopsy instrument, a catheter, or the like. The terms “medical device”, “medical instrument”, “instrument”, “device”, “surgical instrument”, and the like can be used interchangeably herein.

As used herein, the term “electrode” can refer to a specific type of medical device configured for recording and/or stimulation. The terms “electrode”, “depth electrode”, and “intracranial electrode” can be used interchangeably herein.

As used herein, the term “treatment” can refer to a therapy administered to treat a medical condition. Treatments can include electrical stimulation, chemical stimulation, optic stimulation, radiofrequency ablative procedures, laser ablative procedures, radiation ablative procedures, and/or any attempt to modulate a neuronal network.

As used herein, the term “subject” can refer to any warm-blooded organism including, but not limited to, a human being, a pig, a rat, a mouse, a dog, a cat, a goat, a sheep, a horse, a monkey, an ape, a rabbit, a cow, etc. The terms “patient” and “subject” can be used interchangeably herein.

### II. Overview

The present disclosure relates generally to stereo-electroencephalography (SEEG). SEEG is a minimally invasive technique, which allows for recording EEG signals from within the brain using depth electrodes. The recordings can be used to map neuronal networks involved in seizures with the minimal number of depth electrodes. Accordingly, the location of the depth electrodes is of the utmost importance. Currently, there is no standard by which to define such a hypothesis and no specialized intracranial electrodes that exist to facilitate investigation of the hypothesis. As such, the present disclosure relates, more specifically, to systems and methods for configuring a SEEG procedure.

The SEEG procedure can be configured to target specific brain regions according to one or more hypothesis related to a certain neurological disorder. The hypothesis sets forth a hypothetical large-scale neural network responsible for the certain neurological disorder. The hypothesis can be defined based on electro-clinical data from a subject, imaging data of the subject, and neurophysiological data from the subject. Based on the hypothesis, coordinates for implantation of at least two depth electrodes in the patient's brain can be determined. Specific depth electrodes can be selected for each of the coordinates. The depth electrodes can be selected from the group including an insula electrode, a parietal-frontal electrode, a hippocampo electrode, or a perisylvian electrode. In some instances, trajectories for implantation can also be defined. A visualization including the coordinates and the selected electrodes and, in some instances, the trajectories, can be displayed. This enables a surgeon to implant the selected electrodes into the patient's brain according to the coordinates to test the hypothesis. The automated configuration provides a standard SEEG procedure for patients without variability due to the individual surgeon.

### III. Systems

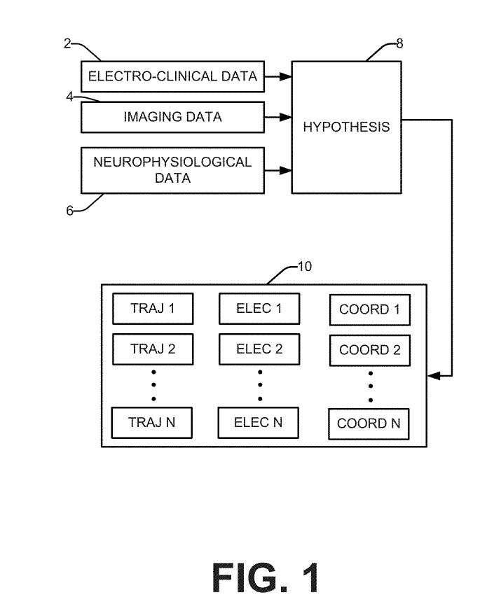

One aspect of the present disclosure can include a system that configures a stereo-electroencephalography procedure to target specific brain regions that are part of large-scale neural networks responsible for neurological disorders, as shown in FIG. 1. The neurological disorder can, for example, be related to at least one of epileptic activity, neuropsychological and/or psychiatric behavior, or abnormalities related to motor function, sensory function, language function, behavior function, memory, vision, or high-level cognitive function. The large-scale neural network can be responsible for modulating and organizing specific human behaviors related to the specific neurological disorder. When the specific brain regions are targeted as being part of the large-scale neuronal networks, a treatment can be performed on areas related within the targeted specific brain regions. The treatment can be related to electrical stimulation, chemical stimulation, optic stimulation, radiofrequency stimulation, laser or radiation ablative procedures, and/or any attempt to modulate the described neuronal network.

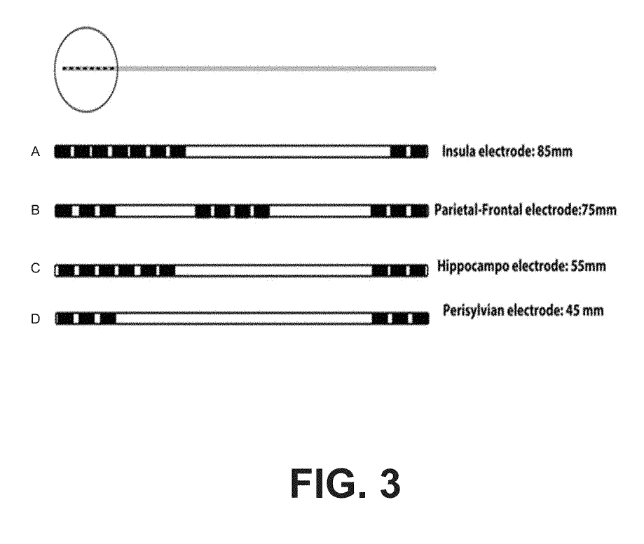

The system can receive and/or define a hypothesis 8 related to a large-scale neural network responsible for a neurological disorder within a patient's brain. The hypothesis 8 can be based on electro-clinical data 2, imaging data 4, and/or neuropsychological data 6. Based on the hypothesis 8, implantation information can be determined. The implantation information can include coordinates (COORD 1-N, where N is 2 or more), electrodes (ELEC 1-N), and trajectories (TRAJ 1-N). The coordinates can be determined based on the hypothesis 8. The coordinates can be expressed in three dimensions in a format that corresponds to the stereotaxic space of the stereotactic system being utilized. An electrode can be selected for each of the coordinates. For each coordinate, the electrode can be an insula electrode (FIG. 3, a), a parietal-frontal electrode (FIG. 3, b), a hippocampo electrode (FIG. 3, c), or a perisylvian electrode (FIG. 3, d). A trajectory can be determined for each of the electrodes from an entry point to the coordinates. A visualization 10 can be displayed including the coordinates, the electrodes, and/or the trajectories.

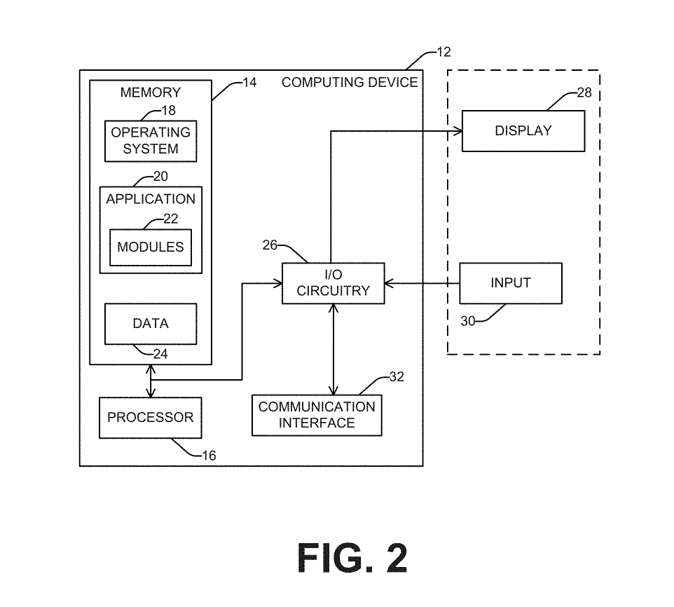

The system shown in FIG. 1 can be implemented at least in part by a computing device. FIG. 2 depicts an example of a computing device 12 that can implement the system shown in FIG. 1. The computing device 12 can include a non-transitory memory 14 and a processor 16. The memory 14 can be a non-transitory memory configured to store machine readable instructions (e.g., operating system 18 and application 20) and/or data 24. The memory 14 could be implemented, for example as volatile memory (e.g., RAM), nonvolatile memory (e.g., a hard disk, flash memory, a solid state drive or the like) or combination of both. The processor 16 (e.g., a processor core) can be configured in the system for accessing the memory 14 and executing the machine-readable instructions. Each element of the system of FIG. 1 can correspond to one or more modules 22 of the application 20. For example, the modules 22 can include (1) a receiving module that receives the hypothesis 8 and/or the electro-clinical data 2, the imaging data 4, and/or the neuropsychological data 6, (2) a determination module, (3) a selection module, and (4) a display module. The computing device 12 can also include I/O circuitry 26, a communication interface 32, an output display 28 (which can be audio or visual) and an input 30. The computing device 12 can refer to a stand-alone device or a network of two or more devices.

The system shown in FIG. 1 provides a systematic way to explore the human cortical and sub-cortical structures using a standard and precise coordinate system with universal electrode types and patient-specific trajectories. The hypothesis 8 can be defined either by the computing device 12 or manually and input to the computing device 12. Coordinates can be determined by consulting a database, which can be stored locally or remotely, that includes coordinates used for other patients corresponding to many different hypotheses. The hypothesis 8 can be matched to a previously-determined hypothesis in the database and the coordinates used to test the previously-determined hypothesis can be output. These coordinates can be used to test the hypothesis 8. The coordinates may have to move because of anatomical features, but the coordinates used previously can provide a good map of the coordinates that should be used to test the hypothesis 8.

The database can also provide the number of electrodes that should be implanted. For example, the number of electrodes should be greater than or equal to 2. However, the in many circumstances, adequate information can be obtained with from 3 to 15 electrodes implanted. The database can also have information related to the specific electrode configuration used to test the previously-determined hypothesis. Notably, the electrodes can be implanted in either an oblique orientation or an orthogonal orientation. The electrodes can also have contacts oriented in a specific manner to gather the most information regarding the neurological condition. The database can also provide information related to the trajectories that were used to deliver the electrode to the coordinate (however, changes to this trajectory can be made based on imaging data 4 related to the patient).

A surgeon can implant the selected electrodes into the patient's brain according to the coordinates and/or according to the trajectories to test the hypothesis 8. A SEEG procedure can be performed with the electrodes implanted in the patient's brain. For example, the SEEG procedure can be used to evaluate intractable epilepsy to provide guidance for surgical planning. With SEEG, the seizure onset zone and areas of spiking can be identified by electrodes in different areas in the patient's head within and around epileptic lesions and areas suspected to be part of the seizure network.

### IV. Methods

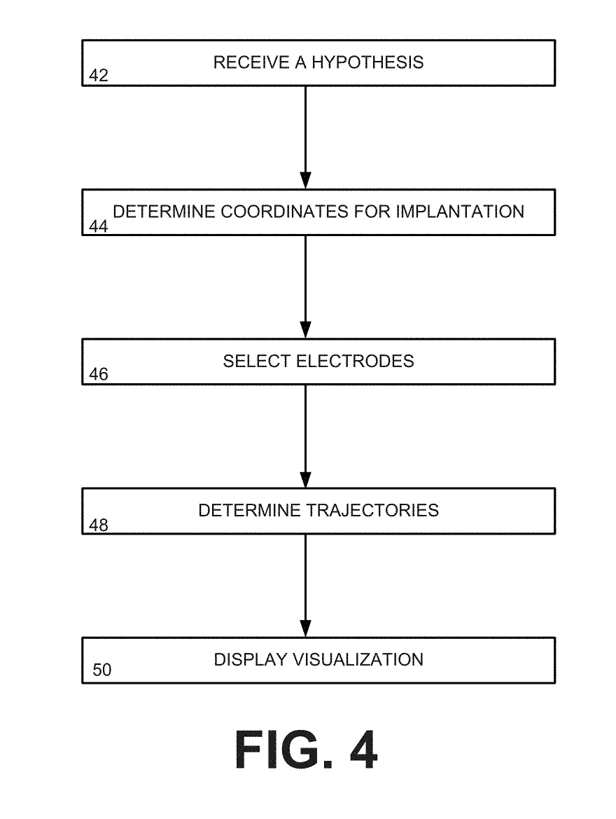

Another aspect of the present disclosure can include a method for configuring a stereo-electroencephalography procedure to target specific brain regions that are part of large scale neural networks responsible for neurological disorders, as shown in FIG. 4. The method is illustrated as process flow diagrams with flowchart illustrations. For purposes of simplicity, the method is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method. The method can be implemented by at least a portion of the computer system shown in FIG. 2.

At 42, a hypothesis can be received. The hypothesis can be related to a large-scale neural network responsible for a neurological disorder within a patient's brain. The neurological disorder can be related to at least one of epileptic activity, neuropsychological and/or psychiatric behavior, or abnormalities related to motor function, sensory function, language function, behavior function, memory, vision, or high level cognitive function. The hypothesis can be based on at least one of electro-clinical data, imaging data, and neuropsychological data. For example, the electro-clinical data, imaging data, and neuropsychological data can be correlated together to study corresponding times related to the ictal development.

At 44, coordinates for implantation of at least two depth electrodes in the patient's brain can be determined. The coordinates can be determined for any number of electrodes greater than two. However, it is more practical to determine coordinates for less than 30 electrodes. In other instances, the coordinates from 3 to 15 depth electrodes can be determined. The coordinates can be determined based on the hypothesis. For example, based on the hypothesis, a database of previous hypotheses and corresponding electrode implantations can be consulted for coordinates that can be used for exploring the hypothesis. The hypothesis can be matched to a similar hypothesis stored in the database, and the coordinates used for the similar hypothesis can be output from the database. The coordinates can be expressed in stereotaxic space depending on the type of stereotactic device being used for the SEEG procedure. For example, the stereotaxic space utilizes a Talairach coordinate system.

At 46, electrodes to be implanted at each of the coordinates can be selected. The electrodes can be selected from a group including insula electrodes, parietal-frontal electrodes, hippocampos electrode, or perisylvian electrodes. Each of the insula electrodes, parietal-frontal electrodes, hippocampos electrode, or perisylvian electrodes can be specially designed for the application related to the coordinates. As an example, the insula electrode can have an oblique orientation and a length of 85 mm with 7 contacts in a distal end and two additional contacts at a proximal end; the parietal-frontal electrode can have an orthogonal orientation and a length of 75 mm with 3 contacts in the distal end, 4 contacts in a middle portion, and an additional 3 contacts at the proximal end; the hippocampo electrode can have an orthogonal orientation and a length of 55 mm with 6 contacts in the distal end and 3 contacts in the proximal end; and the perisylvian electrode can have an orthogonal orientation and a length of 45 mm with 3 contacts in the distal end and 3 contacts in the proximal end

At 48, trajectories for the implantation of the electrodes from a burr hole in the scalp to the coordinates can be determined. In some instances, the trajectories can be determined to minimize complications based on one or more images of the patient's brain. At 50, a visualization can be displayed. The visualization can include at least one of the coordinates, the selected electrodes, and the trajectories. A surgeon can implant the selected electrodes into the patient's brain according to the coordinates to test the hypothesis.

### V. Example

The following example is shown for the purpose of illustration only and is not intended to limit the scope of the appended claims. This example demonstrates the use of stereo-electroencephalography (SEEG) to identify specific brain regions that are part of a large-scale neuronal network responsible for modulating and organizing a seizure disorder. This example shows the feasibility of automating the procedure for determining hypotheses, implantation coordinates and the selected electrodes, as well as the trajectories.

### Hypothesis Development

Before conducting a SEEG procedure, a hypothesis must be determined and expressed. The hypothesis can represent a seizure in neuronal network anatomy rather than cortical anatomy. The hypothesis can also include a three-dimensional conceptualization of the neuronal network. The hypothesis can be based on electro-clinical data from a subject, imaging data of the subject, and/or neurophysiological data from the subject. General principles for hypotheses generation are falsifiability and order of probability. The main hypothesis must be falsifiable by the others of lower order and vice-versa. The process requires generation of alternatives in making clear in anatomical terms the diverse interpretations of the electro-clinical data, the imaging data, and the neurophysiological data. The best way to organize the data is to sort the data by its specific place in the Epileptogenic Process. Therefore electro-clinical data allied with ictal SPECT depict the ictal state/network whereas EEG/MEG and PET the inter-ictal state, and MRI, EEG and PET the lesional area. So, the hypotheses do not only infer the possible ictal networks but also their relations with a MRI or PET image as well. Therefore, electrode implantation implements the whole hypothetical framework in stereotaxic space.

For example, EEG transition from interictal to ictal activities can be evident or subtle, focal or widespread. The evolution of single then rhythmic EEG events, their change in spatial extent, will be carefully estimated with time relation to the parallel occurrence of the string of symptoms and signs. Positive as well as negative elements contribute to disambiguate clinical semiology. For instance, a seizure starting with an ascending epigastric sensation associated with fear, followed by oral and hand automatisms, then loss of contact and orientation of head to one side, will be differently interpreted if clinical onset is delayed by 5 to 10 seconds from an EEG onset characterized by arrest of interictal spikes and flattening, evolving to rhythmic delta-theta pattern in a unilateral temporal region lately spreading to central and fronto-parietal regions; or if clinical onset (made of non-ascending but stationary and intense epigastric sensation, with mastication and drooling immediately followed by dystonic arm posture) is simultaneous with temporal-central discharge spreading to frontal and/or parietal region with vertex electrodes involved. In the first case, the main hypothesis will be that of medial temporal onset with propagation to insular-opercular region, whereas in the second case the reverse trajectory will be the actual one, i.e. insular-opercular onset spreading to neighboring areas and to ipsilateral temporal lobe.

### Implantation Plan

An implantation plan can be designed according to the hypothesis and the three dimensional conceptualization of the network. Even though in the two cases described above the same network should be implanted, the number of electrodes underlying spatial resolution of recording will be weighed depending on the hypothesis order. In the first case, temporal electrodes will include temporal pole, amygdala, anterior and posterior hippocampos, entorhinal and parahippocampal cortex, and perisylvian electrodes superior temporal gyrus/anterior ventral insula, and posteriorly in Supra marginal gyrus/posterior cingulate gyrus. In the second case, three electrodes (amygdala, intermediate hippocampos and temporal pole) will be sufficient, but the perisylvian implantation will be more granular with three or four electrodes in insula, one in orbito-frontal cortex and one in mid-cingulate cortex.

The implantation will target key areas of the hypothetical network, but will also take account of the cortical volume covered, especially in the lateral and ventral cortex. A too focal implantation will be useless, because signal information derived from electrodes located in the same gyrus will be roughly the same and the boundaries of epileptogenic regions are impossible to delineate (because the temporo-spatial ictal dynamics would be impossible to decipher) if some electrodes were not placed remotely in “non-epileptogenic” regions. Two other preoccupations will guide implantation: (i) functional “mapping” and (ii) “sentinel” electrodes to help defining corticectomy limits. For example, in the above cases, if the hypothetical temporal-perisylvian epilepsy is in the dominant hemisphere, some electrodes will be added at the periphery of the core electrodes, i.e, in the posterior superior temporal and supra-marginal gyri as well as in the posterior inferior frontal gyrus on the one hand, in the anterior and mid-cingulate gyrus on the other hand.

The aim to obtain all the possible information from the SEEG exploration should not be pursued at the expense of an excessive number of electrodes, which will likely increase the morbidity of the implantation. In general, implantations that exceed 15 depth electrodes are rare and the requirement of excessive number of electrodes may indicate a loose pre-implantation hypothesis framework. In addition, the possible involvement of “eloquent” regions in the ictal discharge requires their judicious coverage, with the two-fold goal to assess their role in the seizure organization and to define the boundaries of a safe surgical resection.

### Implantation Patterns

SEEG implantation patterns are based on a tailored strategy of exploration that is individualized for each patient. Nevertheless, a number of “typical” patterns of coverage can be a posteriori recognized: (1) Temporal lobe-limbic explorations, (2) Fronto-parietal explorations, (3) Central and pari-central explorations, and (4) Occipital-parietal explorations

(1) Temporal Lobe-Limbic Explorations.

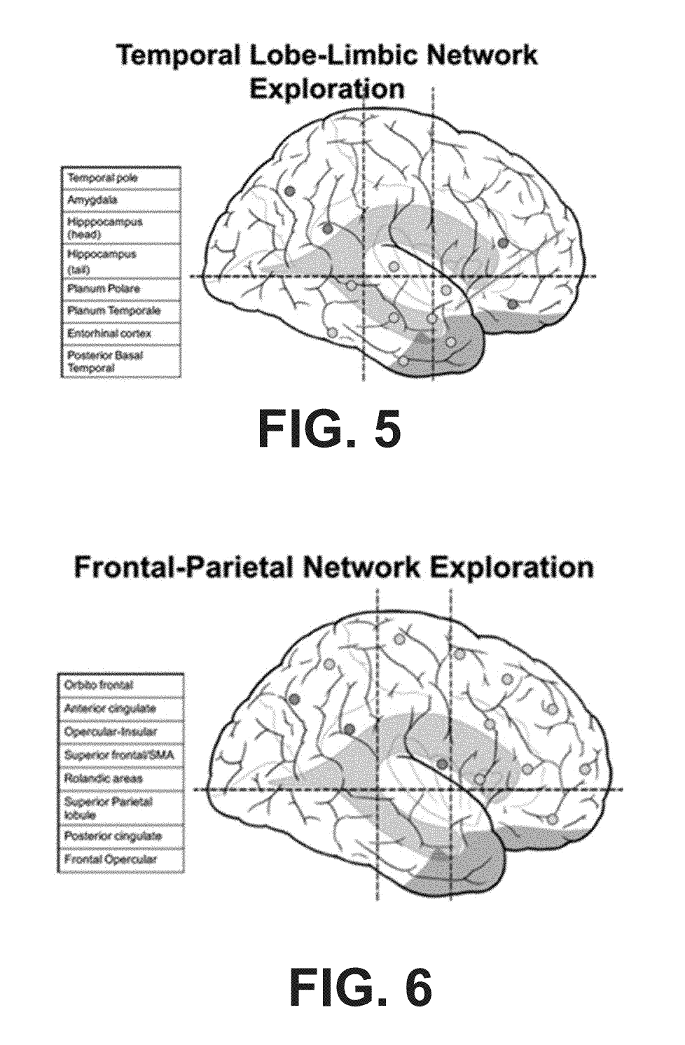

Cases of temporal lobe epilepsy with consistent anatomo-electro-clinical findings suggesting a limbic network involvement are usually operated on after non-invasive investigation only. In general, the use of invasive monitoring is not necessary when semiological and electrophysiological studies demonstrate typical non-dominant mesial temporal epilepsy and imaging studies demonstrate an undisputable MRI lesion (mesial temporal sclerosis, as an example) that fits the initial localization hypothesis. Nevertheless, invasive exploration with SEEG recordings may be required in patients in whom the supposed epileptic zones (EZs) are suspected to involve extra-temporal areas as well. In these cases, the implantation pattern points to disclose a preferential spread of the discharge to the temporo-insular-anterior perisylvian areas, the temporo-insular-orbitofrontal areas, or the posterior temporal, posterior insula, temporo-basal, parietal and posterior cingulate areas. Consequently, sampling of extra-temporal limbic areas must be wide enough to provide information to identify a possible extra-temporal origin of the seizures that could not been anticipated with precision according to non-invasive methods of investigation. An example implantation plan for a temporal lobe-limbic network exploration is shown in FIG. 5.

(2) Fronto-Parietal Explorations.

Due to the large volume of the frontal and parietal lobes, a high number of electrodes are required for an adequate coverage of this region. In most patients, however, excessive sampling can be avoided, and the implantation to more limited portions of the frontal and parietal lobes can be performed. The suspicion of orbito-frontal epilepsy, for instance, often requires the investigation of gyrus rectus, the frontal polar areas, the anterior cingulate gyrus and the anterior portions of the temporal lobe (temporal pole). Similarly, seizures that are thought to arise from the mesial surface of the pre-motor cortex are evaluated by targeting at least the rostral and caudal part of the supplementary motor area (SMA), the pre-SMA area, different portions of the cingulate gyrus and sulcus, as well as the primary motor cortex and mesial and dorsal-lateral aspects of the parietal lobe. Consequently, the hypothesis-based sampling often allows localization of the EZ in the frontal and/or parietal lobes, and in some cases may allow the identification of relatively small EZs. Eventually, frontal-parietal network explorations may be bilateral, and sometimes symmetrical, mainly when a mesial frontal-parietal epilepsy is suspected and the non-invasive methods of investigation failed in lateralizing the epileptogenic process. An example implantation plan for a fronto-parietal network exploration is shown in FIG. 6.

(3) Central and Pari-Central Explorations.

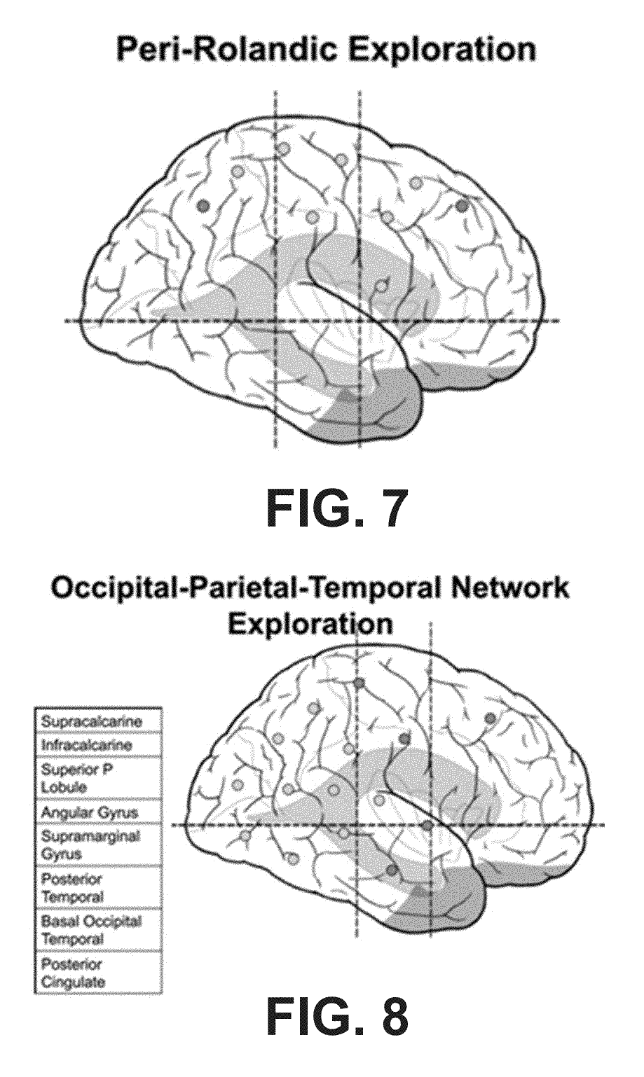

Electrodes in rolandic regions are normally placed when there is a need to define the posterior margin of the resection in frontal explorations or the anterior margin in parietal-occipital explorations, or when the EZ may be located in or near rolandic cortex. The main goal here is to evaluate the rolandic participation to the ictal discharge and to obtain a functional mapping by intracerebral electrical stimulation. In this location, depth electrodes are particularly helpful to sample the depth of the central sulcus, as well as the descending and ascending white matter fibers associated with this region. Also, in many instances, it would be important to explore the immediate adjacent frontal and parietal cortical areas in close anatomical and functional association with the rolandic cortex, as the most caudal portions of the superior frontal gyrus, the pre-central sulcus, the caudal portions of the superior frontal gyrus, the post-central sulcus and the most rostral portions of the superior parietal lobule. In addition, the most central aspect of the cingulate gyrus and sulcus (most possible localization of the cingulate motor areas) should also be targeted. In general, in stereotactic terms, the motor cingulate area is likely located just immediately posterior to vAC (vertical line through anterior commissure), but its precise localization is still a matter of debate. If the rolandic areas in consideration are adjacent to the Sylvian region (perisylvian, probably related to the face motor and sensory cortex), the sub-central gyri should also be targeted, in association with the adjacent insular cortex (middle and posterior short insular gyri). An example implantation plan for a peri-rolandic exploration is shown in FIG. 7.

(4) Occipital-Parietal Explorations.

In the posterior regions, placement of electrodes limited to a single lobe is extremely uncommon, due to the frequent simultaneous involvement of several occipital, parietal and posterior temporal structures, as well as to the multidirectional spread of the discharges to supra and infra-sylvian areas. Consequently, mesial and dorsal lateral surfaces of the occipital lobes are explored, covering both infra-calcarine and supra-calcarine areas, in association with posterior temporal, posterior perisylvian, basal temporal-occipital areas and posterior parietal areas including the posterior inferior parietal lobule and the posterior pre-cuneus. In posterior epilepsies, bilateral explorations are generally needed due to rapid contralateral spread of ictal activity. In addition, in many circumstances, the caudal aspect of the frontal lobe (dorsal-lateral and mesial regions) is also explored with “sentinel” electrodes. An example implantation plan for an occipital-parietal-temporal network exploration is shown in FIG. 8.

### Choice of Electrodes

The electrodes that are implanted into the various regions can be chosen for the particular purpose. Schematic illustrations of four different types of electrodes are shown in FIG. 3.

Insula electrodes (a) include an oblique orientation and have 7 contacts in the distal end (0.5 mm each contact, center-to-center distance 0.75 mm). Additional two contacts at the proximal end. The distance between the two recording areas is 85 mm.

Parietal-frontal electrodes (b) include an orthogonal orientation and are used in parietal-frontal explorations, rolandic/perirolandic explorations, and occipital-parietal-temporal explorations. These electrodes have 3 contacts in the distal end (0.5 mm for each contact, center-to-center distance 0.75 mm), 4 contacts in the middle, and an additional 3 contacts at the proximal end. The distance between the distal and proximal contacts is 75 mm.

Hippocampo electrodes (c) include an orthogonal orientation and are used in limbic/temporal explorations. 7 contacts are in the distal end (0.5 mm for each contact, center-to-center distance 0.75 mm). 3 contacts are in the proximal end. The distance between the distal and proximal contacts is 55 mm.

Perisylvian electrodes (d) include an orthogonal orientation and are used in limbic/temporal explorations, parietal frontal and perirolandic explorations. 3 contacts in the distal end (0.5 mm each contact, center-to-center distance: 0.75 mm). 3 contacts in the proximal end. The distance between distal and proximal contacts is 45 mm.

### SEEG Procedure

Once the SEEG planning is finalized and the electrodes are selected, the electrodes are implanted to their desired target areas using a stereotactic technique through 2.5 mm diameter drill holes using orthogonal or oblique orientation. This allows for intracranial recording from lateral, intermediate or deep cortical and subcortical structures in a three-dimensional arrangement, thus accounting for the dynamic, multidirectional spatiotemporal organization of the epileptogenic pathways.

Individual trajectories are planned within the 3D imaging reconstruction according to predetermined target locations and intended trajectories. Trajectories are selected to maximize sampling from superficial and deep cortical and subcortical areas within the pre-selected zones of interest and are oriented orthogonally in the majority of cases to facilitate the anatomo-electrophysiological correlation during the extra-operative recording phase and to avoid possible trajectories shifts due to excessive angled entry points. Nevertheless, when multiple targets are potentially accessible via a single non-orthogonal trajectory, these multi-target trajectories are selected in order to minimize the number of implanted electrodes per patient. All trajectories are evaluated for safety and target accuracy in their individual reconstructed planes (axial, sagittal, coronal), and also along the reconstructed “probe's eye view”. Any trajectory that appeared to compromise vascular structures is adjusted appropriately without affecting the sampling from areas of interest. A set working distance of 150 mm from the drilling platform to the target are initially utilized for each trajectory, been later adjusted in order to maximally reduce the working distance and, consequently, improve the implantation accuracy. The overall implantation schemas are analyzed using the 3D cranial reconstruction capabilities and internal trajectories are checked to ensure that no trajectory collisions are present. External trajectory positions are examined for any entry sites that would be prohibitively close (less than 1.5 cm distance) at the skin level.

After the SEEG implantation procedure, post-operative images are obtained in order to verify the accuracy of the implantation and define the specific anatomical locations that will be sampled during the seizure semiology analysis and respective interictal and ictal SEEG recording.

From the above description, those skilled in the art will perceive improvements, changes and modifications. Such improvements, changes and modifications are within the skill of one in the art and are intended to be covered by the appended claims.

What is claimed is:

1. A method comprising:
receiving, by a system comprising a processor, a hypothesis related to a large scale neural network responsible for a neurological disorder within patient's brain;
determining, by the system, coordinates for implantation of at least two depth electrodes in the patient's brain based on the hypothesis;
selecting, by the system, electrodes to be implanted for each of the coordinates, wherein the electrodes comprise at least one of an insula electrode, a parietal-frontal electrode, a hippocampo electrode, or a perisylvian electrode; and
displaying, by the system, a visualization comprising the coordinates and the selected electrodes,
wherein a surgeon implants the selected electrodes into the patient's brain according to the coordinates to test the hypothesis.

2. The method of claim 1, further comprising determining, by the system, the hypothesis based on at least one of electro-clinical data, imaging data, and neuropsychological data.

3. The method of claim 1, wherein the determining the coordinates further comprises determining at least one trajectory to reach each of the coordinates,
wherein the trajectory is displayed in the visualization.

4. The method of claim 1, wherein the coordinates are expressed in stereotaxic space

5. The method of claim 4, wherein the stereotaxic space utilizes a Talairach coordinate system.

6. The method of claim 1, wherein the insula electrode has an oblique orientation and a length of 85 mm with 7 contacts in a distal end and two additional contacts at a proximal end,
wherein the parietal-frontal electrode has an orthogonal orientation and a length of 75 mm with 3 contacts in the distal end, 4 contacts in a middle portion, and an additional 3 contacts at the proximal end,
wherein the hippocampo electrode has an orthogonal orientation and a length of 55 mm with 6 contacts in the distal end and 3 contacts in the proximal end, and
wherein the perisylvian electrode has an orthogonal orientation and a length of 45 mm with 3 contacts in the distal end and 3 contacts in the proximal end

7. The method of claim 1, wherein the neurological disorder is related to at least one of epileptic activity, neuropsychological and/or psychiatric behavior, or abnormalities related to motor function, sensory function, language function, behavior function, memory, vision, or high level cognitive function.

8. The method of claim 1, wherein the determining further comprises determining coordinates for implantation of from three to fifteen depth electrodes in the patient's brain based on the hypothesis.

9. A system comprising:
a memory storing instructions; and
a processor to execute the instructions to at least:
receive a hypothesis related to a large scale neural network responsible for a neurological disorder within patient's brain;
determine coordinates for implantation of at least two depth electrodes in the patient's brain based on the hypothesis;
select electrodes to be implanted for each of the coordinates, wherein the electrodes comprise at least one of an insula electrode, a parietal-frontal electrode, a hippocampo electrode, or a perisylvian electrode; and
display a visualization comprising the coordinates and the selected electrodes,
wherein a surgeon implants the selected electrodes into the patient's brain according to the coordinates to test the hypothesis.

10. The system of claim 9, wherein the processor executes the instructions to at least determine the hypothesis based on at least one of electro-clinical data, imaging data, and neuropsychological data.

11. The system of claim 9, wherein the processor executes the instructions to at least determine at least one trajectory to reach each of the coordinates,
wherein the trajectory is displayed in the visualization.

12. The system of claim 9, wherein the coordinates are expressed in stereotaxic space.

13. The system of claim 12, wherein the stereotaxic space utilizes a Talairach coordinate system.

14. The system of claim 9, wherein the insula electrode has an oblique orientation and a length of 85 mm with 7 contacts in a distal end and two additional contacts at a proximal end,
wherein the parietal-frontal electrode has an orthogonal orientation and a length of 75 mm with 3 contacts in the distal end, 4 contacts in a middle portion, and an additional 3 contacts at the proximal end,
wherein the hippocampo electrode has an orthogonal orientation and a length of 55 mm with 6 contacts in the distal end and 3 contacts in the proximal end, and
wherein the perisylvian electrode has an orthogonal orientation and a length of 45 mm with 3 contacts in the distal end and 3 contacts in the proximal end

15. The system of claim 9, wherein the neurological disorder is related to at least one of epileptic activity, neuropsychological and/or psychiatric behavior, or abnormalities related to motor function, sensory function, language function, behavior function, memory, vision, or high level cognitive function.

16. The system of claim 9, wherein the coordinates for implantation are determined for from three to fifteen depth electrodes in the patient's brain based on the hypothesis.
